(19) United States
(12) Patent Application Publication
(10) Pub. No.: US 2014/0037601 A1
(43) Pub. Date: Feb. 6, 2014

(54) NUTRITIONAL COMPOSITIONS INCLUDING BRANCHED CHAIN FATTY ACIDS FOR WOUND HEALING

(71) Applicant: Norman Alan Greenberg, New Hope, MN (US)

(72) Inventor: Norman Alan Greenberg, New Hope, MN (US)

(73) Assignee: NESTEC S.A., Vevey (CH)

(21) Appl. No.: 14/111,668

(22) PCT Filed: Apr. 12, 2012
(86) PCT No.: PCT/EP2012/056645

§ 371 Date: Oct. 14, 2013

Related U.S. Application Data
(60) Provisional application No. 61/474,452, filed on Apr. 12, 2011.

(51) Int. Cl. A61K 31/202; A61K 31/7088; A61K 36/064; A61K 45/06; A61K 36/06; A61K 35/74; A23L 1/30; A61K 38/18

(52) U.S. Cl. CPC A61K 31/202; A23L 1/3008; A61K 31/7088; A61K 38/1841; A61K 45/06; A61K 36/06; A61K 35/745; A61K 35/747; A61K 35/741; A61K 36/064
USPC 424/93.41; 514/560; 514/44 R; 514/8.9; 424/93.5; 424/93.4; 424/93.42; 424/93.44; 424/93.45; 424/93.1; 424/93.51; 424/94.1

(57) ABSTRACT

Nutritional compositions for improving, treating and/or preventing various medical conditions and methods of using same are provided. The medical conditions may include wounds, mucositis among others. The nutritional compositions include branched chain fatty acids and may include other functional ingredients such as, but not limited to probiotics, nucleotides and amino acids. Methods of administering such nutritional products to individuals in need of same are also provided.

### BACKGROUND

The present disclosure relates generally to health and nutrition. More specifically, the present disclosure relates to nutritional compositions having branched chain fatty acids and methods of using same.

There are many types of nutritional compositions currently on the market. Nutritional compositions can be targeted toward certain consumer types, for example, young, elderly, athletic, etc., based on the specific ingredients of the nutritional composition. For example, individuals undergoing cancer treatments such as chemotherapy and/or radiation often experience adverse effects of the treatment. One common side effect is mucositis, which is the painful inflammation and ulceration of the mucous membranes lining the digestive tract. It can occur anywhere along the gastrointestinal (“GI”) tract, but oral mucositis is a common and often debilitating complication of cancer treatment. Other medical conditions that may require nutritional compositions having specific beneficial ingredients may include, for example, allergies, autoimmune diseases, wounds, diabetes, arthritis, cardiovascular disease and stroke, among others.

One goal of nutritional support, therefore, is to provide individuals having an adverse medical condition with nutritional compositions that promote proper healing and/or provide proper treatment or prevention.

### SUMMARY

The present disclosure is related to nutritional compositions having branched chain fatty acids and methods of using same, in particular for use in improving wound healing or reducing the severity of mucositis. In a general embodiment, nutritional compositions including an effective amount of branched chain fatty acids are provided. The nutritional compositions may be formulated for administration to an infant, a child, or an adult.

In an embodiment, methods of improving wound healing in an individual in need of same are provided. The methods include administering to the individual a nutritional composition having an effective amount of branched chain fatty acids. The wound may be a pressure ulcer, burn, surgical trauma, or combinations thereof.

In an embodiment the nutritional composition is formulated for administration to an individual selected from one of an infant, a child, and an adult.

In an embodiment, the nutritional composition is formulated for an infant and the branched chain fatty acids are present in the nutritional composition in an amount from about 0.5% to about 5.0% by weight of total fatty acids. The branched chain fatty acids may also be present in an amount from about 2.0% to about 4.0% by weight of total fatty acids, or about 3.0% by weight of total fatty acids.

In an embodiment, the nutritional compositions are formulated for administration to a child. The nutritional compositions may be administered to the child in an amount that provides branched chain fatty acids in an amount from about 0.5% to about 5.0% by weight of daily total fat or about 2.0% to about 4.0% by weight of total fat.

In an embodiment, the nutritional compositions are formulated for administration to an adult. The nutritional compositions may also be administered to the adult in an amount that provides branched chain fatty acids in an amount from about 500 mg to about 1000 mg per day.

In an embodiment, the nutritional compositions further include a source of ω-3 fatty acids. The source of ω-3 fatty acids may be selected from the group consisting of fish oil, krill, plant sources containing ω-3 fatty acids, flaxseed, walnut, algae, or combinations thereof. The ω-3 fatty acids may be selected from the group consisting of α-linolenic acid (“ALA”), docosahexaenoic acid (“DHA”), stearidonic acid (SDA), eicosapentaenoic acid (“EPA”), or combinations thereof.

In an embodiment, the nutritional compositions further include at least one nucleotide selected from the group consisting of a subunit of deoxyribonucleic acid (“DNA”), a subunit of ribonucleic acid (“RNA”), polymeric forms of DNA and RNA, yeast RNA, or combinations thereof. In an embodiment, the at least one nucleotide is an exogenous nucleotide.

In an embodiment, the nutritional compositions further include a phytonutrient selected from the group consisting of flavanoids, allied phenolic compounds, polyphenolic compounds, terpenoids, alkaloids, sulphur-containing compounds, or combinations thereof. The phytonutrient may be selected from the group consisting of carotenoids, plant sterols, quercetin, curcumin, limonin, or combinations thereof.

In an embodiment, the nutritional compositions further include a source of protein. The source of protein may be selected from the group consisting of dairy based proteins, plant based proteins, animal based proteins, artificial proteins, or combinations thereof. The dairy based proteins may be casein, caseinates, casein hydrolysate, whey, whey hydrolysates, whey concentrates, whey isolates, milk protein concentrate, milk protein isolate, or combinations thereof. The plant based proteins may be soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, or combinations thereof.

In an embodiment, the nutritional compositions further include a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

In an embodiment, the nutritional compositions further include a probiotic selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

In an embodiment, the nutritional compositions further include an amino acid selected from the group consisting of alanine, citrulline, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof. In an embodiment, the amino acid is glutamine. In an embodiment, the amino acid is threonine.

In an embodiment, the nutritional compositions further include an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

In an embodiment, the nutritional compositions further include a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, or combinations thereof.

In an embodiment, the nutritional compositions further include a mineral selected from the group consisting of boron, calcium, chromium, copper, iodine, iron, magnesium, manganese, molybdenum, nickel, phosphorus, potassium, selenium, silicon, tin, vanadium, zinc, or combinations thereof.

In an embodiment, the nutritional compositions are in a form selected from the group consisting of tablets, capsules, liquids, chewables, soft gels, sachets, powders, syrups, liquid suspensions, emulsions, solutions, or combinations thereof.

In an embodiment, the nutritional compositions are oral nutritional supplements. Alternatively, the nutritional compositions may be tube feedings.

In an embodiment, the nutritional compositions are a source of complete nutrition. Alternatively, the nutritional compositions may be a source of incomplete nutrition.

Additional features and advantages are described herein, and will be apparent from the following Detailed Description.

### DETAILED DESCRIPTION

As used herein, “about” is understood to refer to numbers in a range of numerals. Moreover, all numerical ranges herein should be understood to include all integer, whole or fractions, within the range.

As used herein the term “amino acid” is understood to include one or more amino acids. The amino acid can be, for example, alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

As used herein, “animal” includes, but is not limited to, mammals, which include but is not limited to, rodents, aquatic mammals, domestic animals such as dogs and cats, farm animals such as sheep, pigs, cows and horses, and humans. Wherein the terms “animal” or “mammal” or their plurals are used, it is contemplated that it also applies to any animals that are capable of the effect exhibited or intended to be exhibited by the context of the passage.

As used herein, the term “antioxidant” is understood to include any one or more of various substances such as beta-carotene (a vitamin A precursor), vitamin C, vitamin E, and selenium) that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

As used herein, “complete nutrition” includes nutritional products and compositions that contain sufficient types and levels of macronutrients (protein, fats and carbohydrates) and micronutrients to be sufficient to be a sole source of nutrition for the animal to which it is being administered to. Patients can receive 100% of their nutritional requirements from such complete nutritional compositions.

As used herein, “effective amount” is an amount that prevents a deficiency, treats a disease or medical condition in an individual or, more generally, reduces symptoms, manages progression of the diseases or provides a nutritional, physiological, or medical benefit to the individual. A treatment can be patient- or doctor-related.

While the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, sources of ω-3 fatty acids include, for example, fish oil, krill, plant sources of ω-3, flaxseed, walnut, and algae. Examples of ω-3 fatty acids include, for example, α-linolenic acid (“ALA”), docosahexaenoic acid (“DHA”), stearidonic acid (SDA), eicosapentaenoic acid (“EPA”), or combinations thereof.

As used herein, “food grade micro-organisms” means micro-organisms that are used and generally regarded as safe for use in food.

As used herein, “incomplete nutrition” includes nutritional products or compositions that do not contain sufficient levels of macronutrients (protein, fats and carbohydrates) or micronutrients to be sufficient to be a sole source of nutrition for the animal to which it is being administered to. Partial or incomplete nutritional compositions can be used as a nutritional supplement.

As used herein, “long term administrations” are preferably continuous administrations for more than 6 weeks. Alternatively, “short term administrations,” as used herein, are continuous administrations for less than 6 weeks.

As used herein, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

The term “microorganism” is meant to include the bacterium, yeast and/or fungi, a cell growth medium with the microorganism, or a cell growth medium in which microorganism was cultivated.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, a “non-replicating” microorganism means that no viable cells and/or colony forming units can be detected by classical plating methods. Such classical plating methods are summarized in the microbiology book: James Monroe Jay, et al., Modern food microbiology, 7th edition, Springer Science, New York, N.Y. p. 790 (2005). Typically, the absence of viable cells can be shown as follows: no visible colony on agar plates or no increasing turbidity in liquid growth medium after inoculation with different concentrations of bacterial preparations (‘non replicating’ samples) and incubation under appropriate conditions (aerobic and/or anaerobic atmosphere for at least 24 h). For example, bifidobacteria such as Bifidobacterium longum, Bifidobacterium lactis and Bifidobacterium breve or lactobacilli, such as Lactobacillus paracasei or Lactobacillus rhamnosus, may be rendered non-replicating by heat treatment, in particular low temperature/long time heat treatment.

As used herein, a “nucleotide” is understood to be a subunit of deoxyribonucleic acid (“DNA”), ribonucleic acid (“RNA”), polymeric RNA, polymeric DNA, or combinations thereof. It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation. The exogenous nucleotide can be in a monomeric form such as, for example, 5′-Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”), or combinations thereof. The exogenous nucleotide can also be in a polymeric form such as, for example, an intact RNA. There can be multiple sources of the polymeric form such as, for example, yeast RNA.

“Nutritional products,” or “nutritional compositions,” as used herein, are understood to include any number of optional additional ingredients, including conventional food additives (synthetic or natural), for example one or more acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount. The nutritional products or compositions may be a source of complete nutrition or may be a source of incomplete nutrition.

As used herein the term “patient” is understood to include an animal, especially a mammal, and more especially a human that is receiving or intended to receive treatment, as it is herein defined.

As used herein, “phytochemicals” or “phytonutrients” are non-nutritive compounds that are found in many foods. Phytochemicals are functional foods that have health benefits beyond basic nutrition, are health promoting compounds that come from plant sources, and may be natural or purified. “Phytochemicals” and “Phytonutrients” refers to any chemical produced by a plant that imparts one or more health benefit on the user. Non-limiting examples of phytochemicals and phytonutrients include those that are:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and omega-3, 6, and 9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors;

vi) other organic acids which include oxalic acid, phytic acid (inositol hexaphosphate); tartaric acid; and anacardic acid; or

vii) combinations thereof.

As used in this disclosure and the appended claims, the singular forms “a,” “an” and “the” include plural referents unless the context clearly dictates otherwise. Thus, for example, reference to “a polypeptide” includes a mixture of two or more polypeptides, and the like.

As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn R. Gibson and Marcel B. Roberfroid, “Dietary Modulation of the Human Colonic Microbiota: Introducing the Concept of Prebiotics,” J. Nutr., 125: 1401-1412 (1995). Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof.

As used herein, probiotic micro-organisms (hereinafter “probiotics”) are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al., “Probiotics: how should they be defined?,” Trends Food Sci. Technol., 10, 107-10 (1999). In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

Non-limiting examples of proteins include dairy based proteins, plant based proteins, animal based proteins and artificial proteins. Dairy based proteins may be selected from the group consisting of casein, caseinates, casein hydrolysate, whey, whey hydrolysates, whey concentrates, whey isolates, milk protein concentrate, milk protein isolate, or combinations thereof. In a preferred embodiment, TGF-β, in particular in the form of TGF-beta2, may be added to the formula in the form of a whey protein fraction enriched in these bioactive peptides such as TM0301 or XP-828L from Armor Protéines, France or in the form of a polypeptide growth factor isolated from milk as described for example in EP 313515 or WO 92/00994, or in the form of casein rich in TGF-beta2 as in EP1420811.

Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, and any proteins derived from beans, buckwheat, lentils, pulses, single cell proteins, or combinations thereof. Animal based proteins may be selected from the group consisting of beef, poultry, fish, lamb, seafood, or combinations thereof.

All dosage ranges contained within this application are intended to include all numbers, whole or fractions, contained within said range.

As used herein, a “synbiotic” is a supplement that contains both a prebiotic and a probiotic that work together to improve the microflora of the intestine.

As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

As used herein, a “tube feed” is a complete or incomplete nutritional product or composition that is administered to an animal's gastrointestinal system, other than through oral administration, including but not limited to a nasogastric tube, orogastric tube, gastric tube, jejunostomy tube (“J-tube”), percutaneous endoscopic gastrostomy (“PEG”), port, such as a chest wall port that provides access to the stomach, jejunum and other suitable access ports.

As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) essential in minute amounts for normal growth and activity of the body and obtained naturally from plant and animal foods or synthetically made, pro-vitamins, derivatives, analogs.

The present disclosure is related to nutritional compositions having branched chain fatty acids (“BCFA”), and methods of using same. BCFAs are essential saturated fatty acids with one or more methyl branches on the carbon chain. The most common branched chain fatty acids are mono-methyl-branched, but di- and poly-methyl-branched fatty acids are also known. In mono-methyl BCFA, the major branching is at the terminal methyl (iso) or next to the terminal methyl (anteiso). Iso- and anteiso-BCFA are the main BCFA reported in cow's milk. Their main function in cell membranes may be to increase the fluidity of lipids as an alternative to double bonds, which are more liable to oxidation.

BCFAs are typically not found in synthetic, refined formulations. Instead, BCFAs are synthesized by the skin and are known components of the vernix caseoa in utero. The vernix, which is the waxy white substance found coating the skin of newborn human babies, may be ingested in utero and metabolized by the fetal GI tract. See, “Branched Chain Fatty Acids Are Constituents of the Normal Healthy Newborn Gastrointestinal Tract,” Ped. Res., 64(6): 605-609 (2008). Additionally, BCFAs are found in human breast milk at several-fold the concentration of other fatty acids such as docosahexaenoic acid (“DHA”) and arachidonic acid (“ARA”), and also appear in normal diet from cow's milk, cheese and beef. See, “Branched Chain Fatty Acid Content of United States Retail Cow's Milk and Implications for Dietary Intake,” Lipids, February 4-In Press (2011). Currently there are no dietary recommendations for the intake of BCFAs.

Although lipids provide an important energy source in the diet, BCFAs are a specialized class of lipids that serve as bioactives to enhance a host of physiological functions. Indeed, these essential lipids become especially important during various medical conditions and/or disease states. For example, BCFAs may be used to help with wound healing related to conditions including, but now limited to, pressure ulcers and burns, and may be beneficial for reducing the severity of mucositis experienced by cancer patients undergoing therapy. Mucositis is the painful inflammation and ulceration of the mucous membranes lining the digestive tract, usually as an adverse effect of chemotherapy and radiotherapy treatment for cancer. Mucositis can occur anywhere along the GI tract, but oral mucositis refers to the particular inflammation and ulceration that occurs in the mouth. Oral mucositis is a common and often debilitating complication of cancer treatment.

BCFAs may also be synergistically combined with other functional ingredients to provide enhanced physiologic affects upon ingestion. For example, the combination of glutamine and BCFA may provide an enhanced benefit for healing of the mucosal tissue due to damage by cancer treatment. Alternatively, the addition of nucleotides may also act synergistically to improve the healing of mucosal tissue, especially in the small intestine where there is very limited synthesis of nucleotides by the epithelial tissue.

Neurocognitive development and function in individuals from infants to the elderly may also be improved by administration of compositions having BCFAs. In this manner, Applicant believes that there may be a possible interrelationship between vitamin B12 deficiency (e.g., as found with aging) and abnormal fatty acids profiles (increased odd chain fatty acids) of brain cerebral sphingolipids. See, “Fatty Acid Composition of Myelin Isolated From the Brain of a Patient With Cellular Deficiency of Co-enzyme Forms of Vitamin B12,” J. Neurol. Sci. 34(2): 221-32 (1977).

BCFAs may also represent a “good” saturated fat with essential elements for normal physiological function.

Gut barrier function in response to infection (e.g., sepsis), malabsorption/allergy, irritable bowel syndrome (IBS), inflammatory bowel disease (“IBD”) and diarrhea (including, for example, osmotic and/or antibiotic-associated) may also be improved upon administration and ingestion of BCFAs, which may be incorporated into phospholipids. Although accounting for only a minor part of the GI mucus, phospholipids are indispensable in the maintenance of an intact barrier function. Furthermore, BCFAs specifically reduce intestinal permeability through increased tight junction assembly of the transmembrane proteins zonna occludens-2, occludin and claudin-1. As such, BCFAs may be used to support the maintenance of GI tract integrity both in the small and the large bowel.

Further, amino acids such as threonine, proline or serine are a major component of the mucus or mucin that coats the luminal surface. By providing a combination of BCFAs and threonine (or proline or serine), the two act to further enhance gut barrier. The further addition of other amino acids such as glutamine, which is a preferred fuel for enterocytes, further improves the small bowel integrity.

The structure and function of the small bowel can be even further enhanced by inclusion of nucleotides as free or polymeric nucleotides (RNA or other forms). The small intestine is benefited by the inclusion of nucleotides due to its limited ability to synthesize nucleotides de novo and dependence on recycling of nucleotides via salvage pathways. During metabolic stress, even the salvage pathways may be compromised due to a lack of available energy to move molecules through the process of salvage.

The structure and function of the small and large bowel can be even further enhanced by the provision of high quality protein such as whey, which can provide the anabolic signals needed to favor anabolism in both the smooth muscle that supports both the small and large bowel. Large quantities of whey protein can be provided by use of whey protein hydrolysates and/or whey protein micelles.

A further benefit for the large bowel can be achieved by the provision of prebiotic fibers to support the growth of favorable bacteria such that the microbiota will be comprised of a higher percentage of lactobacilli, Bifidis and other bacteria that provide benefit to the large bowel. This benefit can be delivered via the production in situ of short chain fatty acids that the colonocytes metabolize for energy or, more directly, by interaction with the luminal wall to manage the level of inflammation so that destructive levels of inflammatory cytokines are not present. The favorable bacteria may also provide signaling to the host via toll like receptors (“TLR”) which allow for the proper tolerance by the host (mammal) so that the immune system does not try to eliminate the microbiota.

BCFAs are also important with elemental diets as a result of cow's milk induced allergy or severe malabsorption. In this manner, BCFAs help to support normal gut colonization and may be utilized by enterocyte cell membranes. Further, BCFAs demonstrate a synergistic effect with probiotics (including, for example, non-replicating microorganisms) and allow select species (e.g., bifidobacteria, lactobacilli) to be incorporated in the microbiota. The synbiotic relationship of BCFAs plus probiotics demonstrates an unexpected enhancement of pathogen protection in the GI tract.

As mentioned briefly above, BCFAs may also be important for provision to newborns delivered by Caesarian section who avoid the initial stimulus for development of the innate immune system triggered via vaginal delivery. In this manner, vernix suspended in amniotic fluid can be swallowed by a late term fetus. Since BCFAs are synthesized by the skin and are known components of the vernix caseoa in utero, the vernix ingested in utero may be metabolized by the fetal GI tract and help to provide a favorable modulation of the Th1/Th2 response. BCFAs may be metabolized by a distinct pathway and not via beta-oxidation. BCFAs are, therefore, preferentially incorporated in subclasses of lipids and not oxidized for energy.

As an additional benefit, BCFAs may also function as a prebiotic. The ability to affect the microbiota will change the interaction with the host to cause a shift in the Th1:Th2 ratio that can down-regulate the immune system to reduce autoimmune system. For example, eczema is a skin condition caused by an autoimmune reaction. Traditional prebioitcs such as fibers have been shown to have a positive effect on eczema severity. Applicant has found, however, that the use of BCFAs alone, and in combination with prebiotics, may have an enhanced benefit for reduction of the severity of eczema as compared to traditional fiber prebiotics used alone for this benefit. These prebiotic combinations can be further combined with probiotics which can be live and active or non-replicating microorganisms to provide a further synergistic benefit for autoimmune conditions such as eczema.

The nutritional compositions of the present disclosure may include BCFAs in effective amounts. The amount of BCFAs may be dependent upon the individual that is administered the nutritional compositions (e.g., an infant, a child, an adult, the elderly, etc.). As used herein, an “infant” is about 12 months of age or less, a “child” is about one year of age to about 13 years of age, and an “adult” is over about 13 years of age. The amount of BCFAs may also be administered in a bolus or in several smaller doses so as to administer a specific amount of BCFAs per day.

For example, in an embodiment, the nutritional compositions are formulated for consumption by an infant (e.g., infant formula). In such nutritional compositions, BCFAs may be present in an amount of about 0.5 to about 5.0% by weight of the total fatty acids. In another embodiment, the BCFAs are present in the infant composition from about 1.0 to about 4.0% by weight of the total fatty acids. In another embodiment, the BCFAs are present in the infant composition from about 2.0 to about 3.0% by weight of the total fatty acids. In another embodiment, the BCFAs are present in the infant composition in an amount of about 2.5% by weight of the total fatty acids.

The present nutritional compositions may also be formulated for consumption by a child. In such nutritional compositions, BCFAs may be present in an amount of about 0.5% to about 5.0% of the child's total fat intake per day. In another embodiment, the BCFAs are present in the child's nutritional composition in an amount from about 1.0 to about 4.0% of the child's total fat intake per day. In another embodiment, the BCFAs are present in the child's nutritional composition in an amount from about 2.0 to about 3.0% of the child's total fat intake per day. Alternatively, the nutritional compositions may provide the child with about 100 to about 500 mg BCFA per day, or about 200 to about 400 mg BCFA per day, or about 300 mg BCFA per day.

In nutritional compositions formulated for adults and/or therapeutic dosing, the nutritional compositions may be administered so as to provide the adult or patient with about 500 to about 1,000 mg BCFA per day. In another embodiment, the nutritional compositions may be administered so as to provide the adult or patient with about 700 to 800 mg BCFA per day, or about 600 BCFA per day.

The nutritional compositions of the present disclosure may be administered by any means suitable for human administration, and in particular for administration in any part of the gastrointestinal tract. Enteral administration, oral administration, and administration through a tube or catheter are all covered by the present disclosure. The nutritional compositions may also be administered by means selected from oral, rectal, sublingual, sublabial, buccal, topical, etc.

If the nutritional compositions are formulated to be administered orally, the compositions may be a liquid oral nutritional supplement (e.g., incomplete feeding) or a complete feeding. In this manner, the nutritional compositions may be administered in any known form including, for example, tablets, capsules, liquids, chewables, soft gels, sachets, powders, syrups, liquid suspensions, emulsions and solutions in convenient dosage forms. In soft capsules, the active ingredients are preferably dissolved or suspended in suitable liquids, such as fatty oils, paraffin oil or liquid polyethylene glycols. Optionally, stabilizers may be added.

The nutritional compositions of the present disclosure may be a source of either incomplete or complete nutrition. The nutritional compositions may also be used for short term or long term tube feeding.

Suitable nutritional composition formats according to the present disclosure include, for example, infant formulas, solutions, ready-for-consumption compositions (e.g. ready-to-drink compositions or instant drinks), liquid comestibles, soft drinks, juice, sports drinks, milk drinks, milk-shakes, yogurt drinks, soup, etc. In a further embodiment, the nutritional compositions may be manufactured and sold in the form of a concentrate, a powder, or granules (e.g. effervescent granules), which are diluted with water or other liquid, such as milk or fruit juice, to yield a ready-for-consumption composition (e.g. ready-to-drink compositions or instant drinks).

As described herein above, Applicant has surprisingly found that nutritional compositions such as, for example, oral nutritional supplements and/or enteral formulas comprising BCFAs as an essential saturated fat can help to promote improved GI function, wound healing, mucositis among other benefits.

The nutritional compositions may include a source of ω-3 and/or ω-6 fatty acids. Examples of sources of ω-3 fatty acids include, for example, fish oil, krill, plant sources of ω-3, flaxseed, walnut, and algae. Non-limiting examples of ω-3 fatty acids include α-linolenic acid (“ALA”), docosahexaenoic acid (“DHA”), stearidonic acid (SDA) and eicosapentaenoic acid (“EPA”). Non-limiting examples of ω-6 fatty acids include linoleic acid (“LA”), arachidonic acid (“ARA”).

In an embodiment, the nutritional compositions include a source of phytochemicals. Phytochemicals are non-nutritive compounds that are found in many fruits and vegetables, among other foods. There are thousands of phytochemicals that can be categorized generally into three main groups. The first group is flavonoids and allied phenolic and polyphenolic compounds. The second group is terpenoids, e.g., carotenoids and plant sterols. The third group is alkaloids and sulfur containing compounds. Phytochemicals are active in the body and, in general, act similarly to antioxidants. They also appear to play beneficial roles in inflammatory processes, clot formation, asthma, and diabetes. Researchers have theorized that to receive the most benefit from consumption of phytochemicals, they should be consumed as part of whole foods, because of the complex, natural combination and potentially synergistic effects. This may partially explain the health benefits associated with consumption of whole fruits and vegetables. Increased intake of fruits and vegetables is associated with reduced risk of many chronic diseases. In order to enhance the phytochemical profile of the present nutritional compositions, in an embodiment, the compositions include various fruits and vegetables containing these compounds.

In an embodiment, the nutritional compositions include a source of protein. The protein source may be dietary protein including, but not limited to animal protein (such as milk protein, meat protein or egg protein), vegetable protein (such as soy protein, wheat protein, rice protein, and pea protein), or combinations thereof. In an embodiment, the protein is selected from the group consisting of whey, chicken, corn, caseinate, wheat, flax, soy, carob, pea or combinations thereof. In another embodiment, the protein is pea protein or pea protein isolate.

In an embodiment, vegetable proteins will be included to further enhance the net alkaline profile of the formula and increase the variety of macronutrient sources. Based on the nutritional profile of specific vegetable proteins (e.g., pea protein isolate) there are limitations in the amount of vegetable protein sources that can be included in a formula. For example, the amino acid profile of pea protein includes all of the indispensable amino acids. Pea protein is relatively rich in arginine, but limiting in the sulphur-containing amino acids, methionine, and cysteine. However, it is possible, for example, to blend pea protein isolates with a complete protein source (such as milk protein or complete vegetable proteins) having sufficient sulphur-containing amino acids to offset such deficiency. Canola protein (i.e., isolates, hydrosylates and concentrates) is one such vegetable protein which can provide appreciable amounts of sulfur-containing amino acids to further augment the amino acid profile to deliver the necessary protein quality to the patient. Additionally, animal derived proteins are typically more abundant in sulphur-containing amino acids than vegetable proteins.

In an embodiment, the nutritional compositions of the present disclosure are lactose free and/or gluten free.

The nutritional compositions of the present disclosure may also include a source of carbohydrates. Any suitable carbohydrate may be used in the present nutritional compositions including, but not limited to, sucrose, lactose, glucose, fructose, corn syrup solids, maltodextrin, modified starch, amylose starch, tapioca starch, corn starch or combinations thereof.

The nutritional compositions may also include grains. The grains may include, for example, whole grains, which may be obtained from different sources. The different sources may include semolina, cones, grits, flour and micronized grain (micronized flour), and may originate from a cereal or a pseudo-cereal. In an embodiment, the grain is a hydrolyzed whole grain component. As used herein, a “hydrolyzed whole grain component” is an enzymatically digested whole grain component or a whole grain component digested by using at least an alpha-amylase, which alpha-amylase shows no hydrolytic activity towards dietary fibers when in the active state. The hydrolyzed whole grain component may be further digested by the use of a protease, which protease shows no hydrolytic activity towards dietary fibers when in the active state. The hydrolyzed whole grain component may be provided in the form of a liquid, a concentrate, a powder, a juice, a puree, or combinations thereof.

A source of fat may also be included in the present nutritional compositions. The source of fat may include any suitable fat or fat mixture. For example, the fat source may include, but is not limited to, vegetable fat (such as olive oil, corn oil, sunflower oil, high-oleic sunflower, rapeseed oil, canola oil, hazelnut oil, soy oil, palm oil, coconut oil, blackcurrant seed oil, borage oil, lecithins, and the like), animal fats (such as milk fat), or combinations thereof. The source of fat may also be less refined versions of the fats listed above (e.g., olive oil for polyphenol content).

In an embodiment, the nutritional compositions further include one or more prebiotics. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

The nutritional compositions may further include one or more probiotics. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

One or more amino acids may also be present in the nutritional compositions. Non-limiting examples of amino acids include alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

One or more antioxidants may also be present in the nutritional compositions. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

The nutritional compositions also include fiber or a blend of different types of fiber. The fiber blend may contain a mixture of soluble and insoluble fibers. Soluble fibers may include, for example, fructooligosaccharides, acacia gum, inulin, etc. Insoluble fibers may include, for example, pea outer fiber.

The nutritional compositions of the present disclosure may be a source of either incomplete or complete nutrition. The nutritional compositions may be administered by oral administration or tube feeding. If the nutritional compositions are formulated to be administered orally, the compositions may be a liquid oral nutritional supplement or feeding. The nutritional compositions may also be used for short term or long term tube feeding.

Methods of administering the nutritional compositions of the present disclosure are provided. In an embodiment, methods of improving wound healing in an individual in need of same are provided. The methods include administering to the individual a nutritional composition having an effective amount of branched chain fatty acids. The wound may be, for example, a pressure ulcer, burn, surgical trauma, or combinations thereof.

In yet another embodiment, methods of reducing the severity of mucositis in an individual in need of same are provided. The methods include administering to the individual a nutritional composition having an effective amount of branched chain fatty acids.

In an embodiment the nutritional compositions may be formulated for administration to an individual selected from one of an infant, a child, and an adult. In an embodiment, the nutritional compositions are formulated for an infant and the branched chain fatty acids are present in the nutritional composition in an amount from about 0.5% to about 5.0% by weight of total fatty acids. In an embodiment, the nutritional compositions are formulated for administration to a child. The nutritional compositions may be administered to the child in an amount that provides branched chain fatty acids in an amount from about 0.5% to about 5.0% by weight of daily total fat. In an embodiment, the nutritional compositions are formulated for administration to an adult. The nutritional compositions may also be administered to the adult in an amount that provides branched chain fatty acids in an amount from about 500 mg to about 1000 mg per day.

In the methods disclosed herein, the nutritional compositions may be administered through an administration route selected from the group consisting of orally, topically, a tube or catheter, or combinations thereof.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for use in improving wound healing comprising administering a nutritional composition having an effective amount of branched chain fatty acids to an individual in need of same.

2. The method according to claim 1, wherein the wound is selected from the group consisting of a pressure ulcer, burn, surgical trauma and combinations thereof.

3. The method according to claim 1, wherein the wound healing is beneficial for reducing the severity of mucositis or damage of mucosal tissue.

4. The method according to claim 3, wherein mucosal tissue damage or mucositis is experienced by cancer patients undergoing therapy.

5. The method according to claim 1, wherein the composition is formulated for administration to an individual selected from the group consisting of an infant, a child, and an adult.

6. The method according to claim 1, wherein the nutritional composition is formulated for an infant and the branched chain fatty acids are present in the nutritional composition in an amount from about 0.5% to about 5.0% by weight of total fatty acids.

7. The method according to claim 1, wherein the nutritional composition is formulated for administration to a child.

8. The method according to claim 7, wherein the nutritional composition is administered to the child in an amount to provide branched chain fatty acids in an amount from about 0.5% to about 5.0% by weight of total daily fat intake.

9. The method according to claim 7, wherein the nutritional composition is administered to the child in an amount to provide branched chain fatty acids in an amount from about 100 mg to about 500 mg daily.

10. The method according to claim 1, wherein the nutritional composition is formulated for administration to an adult.

11. The method according to claim 10, wherein the nutritional composition is administered to the adult in an amount to provide branched chain fatty acids in an amount from about 500 mg to about 1000 mg daily.

12. The method according to claim 1, wherein the composition comprises a source of ω-3 fatty acids, wherein the source of ω-3 fatty acids is selected from the group consisting of fish oil, hill, plant sources containing ω-3 fatty acids, flaxseed, walnut, algae, and combinations thereof.

13. The method according to claim 12, wherein the ω-3 fatty acids are selected from the group consisting of α-linolenic acid (“ALA”), docosahexaenoic acid (“DHA”), eicosapentaenoic acid (“EPA”), and combinations thereof.

14. The method according to claim 1, wherein the composition comprises at least one nucleotide selected from the group consisting of a subunit of deoxyribonucleic acid (“DNA”), a subunit of ribonucleic acid (“RNA”), polymeric forms of DNA and RNA, yeast RNA, and combinations thereof.

15. The method according to claim 14, wherein the at least one nucleotide is an exogenous nucleotide.

16. The method according to claim 1, wherein the composition comprises a phytonutrient selected from the group consisting of flavanoids, allied phenolic compounds, polyphenolic compounds, terpenoids, alkaloids, sulphur-containing compounds, and combinations thereof.

17. The method according to claim 16, wherein the phytonutrient is selected from the group consisting of carotenoids, plant sterols, quercetin, curcumin, limonin, and combinations thereof.

18. The method according to claim 1, wherein the composition comprises a source of protein, wherein the source of protein is selected from the group consisting of dairy based proteins, plant based proteins, animal based proteins, artificial proteins, and combinations thereof.

19. The method according to claim 18, wherein the dairy based proteins are selected from the group consisting of casein, caseinates, casein hydrolysate, whey, whey hydrolysates, whey concentrates, whey isolates, milk protein concentrate, milk protein isolate, and combinations thereof.

20. The method according to claim 19, wherein the composition comprises TGF-beta.

21. The method according to claim 18, wherein the plant based proteins are selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, and combinations thereof.

22. The method according to claim 1, wherein the composition comprises a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, and combinations thereof.

23. The method according to claim 1, wherein the composition comprises a probiotic selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, and combinations thereof.

24. The method according to claim 1, wherein the composition comprises an amino acid selected from the group consisting of alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, and combinations thereof.

25. The method according to claim 24, wherein the amino acid is glutamine.

26. The method according to claim 24, wherein the amino acid is threonine.

27. The method according to claim 1, wherein the composition comprises an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, and combinations thereof.

28. The method according to claim 1, wherein the composition comprises a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, and combinations thereof.

29. The method according to claim 1, wherein the composition comprises a mineral selected from the group consisting of boron, calcium, chromium, copper, iodine, iron, magnesium, manganese, molybdenum, nickel, phosphorus, potassium, selenium, silicon, tin, vanadium, zinc, and combinations thereof.

30. The method according to claim 1, wherein the nutritional composition is in a form selected from the group consisting of tablets, capsules, liquids, chewables, soft gels, sachets, powders, syrups, liquid suspensions, emulsions, solutions, and combinations thereof.

31. The method according to claim 1, wherein the nutritional composition is an oral nutritional supplement or a tube feeding.

32. The method according to claim 1, wherein the nutritional composition is a source of complete nutrition or of incomplete nutrition.

33. The method according to claim 1, wherein the administration occurs through an administration route selected from the group consisting of orally, topically, a tube or catheter, and combinations thereof.
